(19) United States
(12) Patent Application Publication
(10) Pub. No.: US 2026/0124129 A1
(43) Pub. Date: May 7, 2026

(54) COMPOSITIONS AND METHODS FOR INHIBITING PIGMENTATION

(71) Applicant: Elise A. Olsen, Chapel Hill, NC (US)

(72) Inventor: Elise A. Olsen, Chapel Hill, NC (US)

(21) Appl. No.: 19/114,356

(22) PCT Filed: Sep. 23, 2023
(86) PCT No.: PCT/US2023/074975

§ 371 Date: Mar. 21, 2025

Related U.S. Application Data
(60) Provisional application No. 63/409,563, filed on Sep. 23, 2022.

(51) Int. Cl. A61K 8/49; A61Q 5/08; A61Q 19/02

(52) U.S. Cl. CPC A61K 8/4926; A61K 8/49; A61Q 5/08; A61Q 19/02

(57) ABSTRACT

Disclosed herein are compositions and methods for inhibiting pigmentation of hair or skin. The compositions of the disclosure comprise an effective amount of a compound used for inhibition of pigmentation.

### CROSS-REFERENCE TO RELATED APPLICATIONS

This application claims priority to U.S. Provisional Patent Application No. 63/409,563 filed Sep. 23, 2022, which is incorporated herein by reference in its entirety.

### FIELD

This disclosure relates to compositions and methods for inhibiting pigmentation of hair or skin. The compositions of the disclosure comprise an effective amount of a compound used for inhibition of pigmentation.

### BACKGROUND

An unmet need is the alteration or prevention of pigmentation in the hair or skin. There are conditions where the hair or skin may have too much pigmentation where inhibiting the pigmentation could be of value to patients.

### SUMMARY OF THE INVENTION

In an aspect, the disclosure relates to a method of inhibiting pigmentation of hair, skin, or both, the method comprising administering to a subject in need thereof, a safe and effective amount of a compound described herein. In an embodiment, the compound is an FP receptor antagonist. In another embodiment, the FP receptor antagonist is selected from the group including OBE002, OBE022, and BAY-6672. In another embodiment, the FP receptor antagonist is a compound of formula (II), or a pharmaceutically acceptable sat thereof,

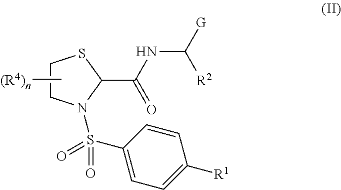

wherein: G is

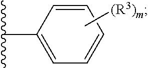

R¹ is tert-butyl or phenyl, wherein the phenyl is optionally substituted with 1-3 R^{1a}; R^{1a}, at each occurrence, is independently C₁₋₄alkyl, C₁₋₄fluoroalkyl, halogen, cyano, —OC₁₋₄alkyl, or —OC₁₋₄fluoroalkyl; R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a}; R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl; R³, at each occurrence, is independently C₁₋₄alkyl, C₁₋₄fluoroalkyl, halogen, cyano, —OC₁₋₄alkyl, or —OC₁₋₄fluoroalkyl; R⁴, at each occurrence, is independently C₁₋₄alkyl or C₁₋₄fluoroalkyl; m is 0, 1, 2, 3, 4, or 5; and n is 0, 1, 2, 3, or 4. In another embodiment, the FP receptor antagonist is a compound of formula (III), or a pharmaceutically acceptable sat thereof,

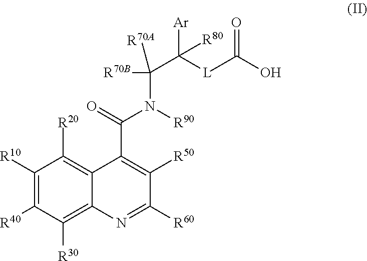

wherein: Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₁₋₄cycloalkyl, —OC₁₋₂alkyl, —SC₁₋₂alkyl, C₁₋₄alkyl substituted by 1-3 fluoro, C₂₋₄cycloakyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy; L is a bond or —X—(CR^{a}R^{b})ₖ—; X is a bond, CH₂, O, S, S(O), S(O)₂, or N(R); R is hydrogen or CH₃; R^{a} and R^{b} are independently hydrogen, fluoro, or CH₃; or R^{a} and R^{b} together with the carbon atom to which they attach form a cyclopropyl; k is 1, 2, 3, or 4; R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₃, —SiCH₃, ethynyl, cyclopropyl, or cyclobutyl, where the cyclopropyl and cyclobutyl are unsubstituted or substituted by 1-4 fluoro; R²⁰, R³⁰, and R⁴⁰ are independently hydrogen, halogen, CH₃, CH₂F, CHF₂, or CF₃; R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro; R⁶⁰ is —NR^{c}R^{d}; R^{c} is hydrogen or C₁₋₃alkyl; R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃; R^{70A} and R^{70B} are independently hydrogen or CH₃, or R^{70A} and R^{70B} together with the carbon atom to which they attach form a cyclopropyl; R⁸⁰ is hydrogen, fluoro, CH₃, CF₃, CH₂CH₃, or OH; and R⁹⁰ is hydrogen or CH₃. In another embodiment, the compound is topically or orally administered to the subject. In another embodiment, pigmentation of hair is inhibited. In another embodiment, pigmentation of skin is inhibited. In another embodiment, the compound is administered for about 6 weeks to about 10 weeks.

In a further aspect, the disclosure relates to a method of lightening skin, the method comprising administering to a subject a safe and effective amount of a compound described herein. In an embodiment, the compound is an FP receptor antagonist. In another embodiment, the FP receptor antagonist is selected from the group including OBE002, OBE022, and BAY-6672. In another embodiment, the FP receptor antagonist is a compound of formula (II), or a pharmaceutically acceptable sat thereof,

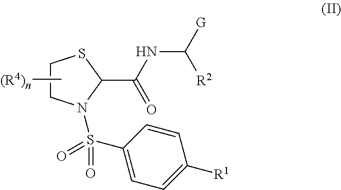

wherein: G is

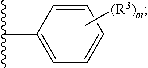

R¹ is tert-butyl or phenyl, wherein the phenyl is optionally substituted with 1-3 R^{1a}; R^{1a}, at each occurrence, is independently C₁₋₄alkyl, C₁₋₄fluoroalkyl, halogen, cyano, —OC₁₋₄alkyl, or —OC₁₋₄fluoroalkyl; R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a}; R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl; R³, at each occurrence, is independently C₁₋₄alkyl, C₁₋₄fluoroalkyl, halogen, cyano, —OC₁₋₄alkyl, or —OC₁₋₄fluoroalkyl; R⁴, at each occurrence, is independently C₁₋₄alkyl or C₁₋₄fluoroalkyl; m is 0, 1, 2, 3, 4, or 5; and n is 0, 1, 2, 3, or 4. In another embodiment, the FP receptor antagonist is a compound of formula (III), or a pharmaceutically acceptable sat thereof,

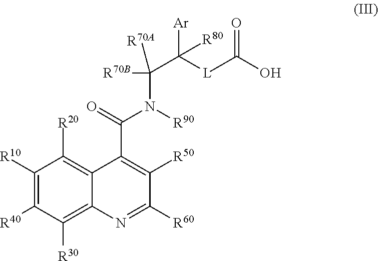

wherein: Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₃₋₄cycloalkyl, —OC₁₋₂alkyl, —SC₁₋₂alkyl, C₁₋₄alkyl substituted by 1-3 fluoro, C₃₋₄cycloakyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy; L is a bond or —X—(CR^{a}R^{b})ₖ—; X is a bond, CH₂, O, S, S(O), S(O)₂, or N(R); R is hydrogen or CH₃; R^{a} and R^{b} are independently hydrogen, fluoro, or CH₃; or R^{a} and R^{b} together with the carbon atom to which they attach form a cyclopropyl; k is 1, 2, 3, or 4; R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro; R²⁰, R³⁰, and R⁴⁰ are independently hydrogen, halogen, CH₃, CH₂F, CHF₂, or CF₃; R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro; R⁶⁰ is —NR^{c}R^{d}; R^{c} is hydrogen or C₁₋₃alkyl; R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃; R^{70A} and R^{70B} are independently hydrogen or CH₃, or R^{70A} and R^{70B} together with the carbon atom to which they attach form a cyclopropyl; R⁸⁰ is hydrogen, fluoro, CH₃, CF₃, CH₂CH₃, or OH; and R⁹⁰ is hydrogen or CH₃. In another embodiment, the compound is administered for about 6 weeks to about 10 weeks. In another embodiment, the subject has melasma, chloasma, postinflammatory hyperpigmentation, lentigines, or hyperpigmentation, or wishes to lighten the skin.

The disclosure provides for other aspects and embodiments that will be apparent in light of the following detailed description.

### DETAILED DESCRIPTION

One object of this disclosure is to provide methods for using compounds to inhibit pigmentation of hair and/or skin. This disclosure relates to compositions comprising compounds to inhibit pigmentation of hair or skin. The disclosure also relates to compostions for inhibiting the formation of pigment in the skin and/or hair, including local or generalized skin with increased pigmentation over normal for that individual, hyperpigmented regions, or skin lightening. The methods as described herein may decrease pigment of the hair and/or skin of a subject.

Publications and patents are referred to throughout this disclosure. All U.S. patents and publications cited herein are hereby incorporated by reference.

All percentages, ratios, and proportions used herein are by weight unless otherwise specified.

In the description, various embodiments and individual features are disclosed. As will be apparent to a person having ordinary skill in the art, all combinations of such embodiments and features are possible and can result in preferred embodiments of the invention.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier“about” used in connection with a quantity is inclusive of the stated value and has the meaning dictated by the context (for example, it includes at least the degree of error associated with the measurement of the particular quantity). The modifier“about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the terms “administering,” “providing” and “introducing” are used interchangeably herein and refer to the placement of the compounds of the disclosure into a subject by a method or route that results in at least partial localization of the compound to a desired site. The compounds can be administered by any appropriate route which results in delivery to a desired location in the subject.

As used herein, the terms “effective amount” or “therapeutically effective amount,” refer to a sufficient amount of an agent or a composition or combination of compositions being administered which will relieve or prevent to some extent one or more of the signs or symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant change (decrease or increase) in condition symptoms, dependent on goal of treatment. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of condition, stage of the condition, route of administration, the type or extent of supplemental therapy used, ongoing condition process and type of treatment desired (e.g., aggressive vs. conventional treatment).

As used herein “increased local or generalized pigmentation” or “hyperpigmented region” means region or regions of the skin having greater than normal pigmentation than the surrounding normal skin. Examples of these include, but are not limited to age spots, lentigines, melasma, chloasma, freckles, photoaging pigmentation changes, post inflammatory hyperpigmentation, post-trauma hyperpigmentation, ultraviolet light-induced pigmented blemishes, sun-induced pigmented blemishes, suntan, and the like. Skin lightening relates to a modification of the current skin color to a lighter shade of pigment.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, refers to a divalent group derived from a straight or branched chain hydrocarbon, for example, of 1 to 3 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). A C₃₋₆ cycloalkyl is monocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “halogen” or “halo,” as used herein, means Cl, Br, I, or F.

Terms such as “alkyl,” “cycloalkyl,” “alkylene,” etc. may be preceded by a designation indicating the number of atoms present in the group in a particular instance (e.g., “C₁₋₄alkyl,” “C₃₋₄cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

“Eflornithine” as used herein refers to a fluoroamino acid that is omithine substituted by a difluoromethyl group at position 2. Eflornithine may also be referred to as 2-difluoromethyl-2,5-diaminopentanoic acid, 2-difluoromethyl-2,5-diaminovaleric acid, or α-(difluoromethyl)ornithine. The abbreviation “DFMO” is employed to refer to α-(difluoromethyl)ornithine.

“Pharmaceutically acceptable” means suitable for use in a human or other mammal.

The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloride, hydrobromide, sulfuric, phosphoric and the like.

“Safe and effective amount” means a quantity of a compound high enough to provide a significant positive modification of the subject's condition or projected condition to be treated, but low enough to avoid serious side effects (at a reasonable benefit/risk ratio).

As used herein, “skin lightening” means decreasing melanin in skin, including one or more of; overall lightening of basal skin tone, lightening of hyperpigmented regions including age spots, lentigines, melasma, chloasma, freckles, photoaging pigmentation changes, post inflammatory hyperpigmentation, suntan, ultraviolet light-induced pigmented blemishes, or sun-induced pigmented blemishes. Skin lightening may also be referred to as whitening, brightening, clarifying, fading, or bleaching.

As used herein, the terms “subject” and “patient” may be used interchangeably to refer to any vertebrate including, but not limited to, a mammal and a human. In some embodiments, the subject may be a human or a non-human. The subject or patient may be undergoing forms of treatment.

As used herein, the term “mammal” refers to any member of the class Mammalia including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

As used herein, the terms “treat,” “treating” or “treatment” are each used interchangeably to describe reversing, alleviating, or inhibiting the progress of a condition and/or injury, or one or more symptoms of such condition, to which such term applies. Depending on the condition of the subject, the term may also refer to preventing a condition, and includes preventing the onset or severity of a condition, or preventing the symptoms associated with a condition. A treatment may be either performed in an acute or chronic way. The term also refers to reducing the severity of a disease or symptoms associated with such condition prior to affliction with the condition. Such prevention or reduction of the severity of a condition prior to affliction refers to administration of a pharmaceutical composition to a subject that is not at the time of administration afflicted with the condition. “Preventing” also refers to preventing the recurrence of a condition or of one or more symptoms associated with such condition. “Treatment” and “therapeutically,” refer to the act of treating, as “treating” is defined above.

### 2. COMPOUNDS

In one aspect, the disclosure relates to compounds for inhibiting pigmentation of hair or skin. In a particular embodiment, the compound may be a polyamine pathway inhibitor, an omithine decarboxylase inhibitor, or polyamine such as any of the compounds as disclosed in U.S. Pat. Nos. 7,374,770; 8,551,462; 7,432,302; 5,811,634; 5,132,293; 6,743,822; 7,015,349; 7,261,878; 5,132,293; 4,720,489; 5,648,394; 7,314,959; 7,045,550; U.S. Patent Application Publication No. 2004/0131574; U.S. Patent Application Publication No. 2004/0006045; U.S. Patent Application Publication No. 2003/0036561; U.S. Patent Application Publication No. 2003/0199584; U.S. Patent Application Publication No. 2003/0185778; International Patent Application Publication No. 2009/052518; International Patent Application Publication No. 2014/080015; and International Patent Application Publication No. 2005/105729, each of which is incorporated herein by reference in their entireties. The omithine decarboxylase inhibitor may be the active ingredient. The ornithine decarboxylase inhibitor may be 2-(difluoromethyl)-2,5-diaminopentanoic acid; alpha-ethynyl omithine; 6-heptyne-2,5-diamine; or 2-methyl-6-heptyne diamine.

The omithine decarboxylase inhibitor may be a compound of Formula (IV), or a pharmaceutically acceptable salt thereof,

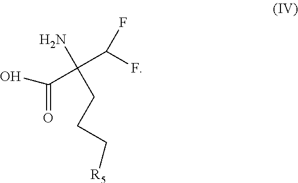

Wherein, R′ is (C₁-C₈)alkoxy, for example, methoxy, ethoxy, isopropoxy, n-propoxy, or tert-butoxy; R₅ is

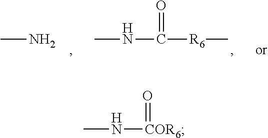

R₆ is phenyl, benzyl, or (C₁-C₄)alkyl.

In an embodiment, the ornithine decarboxylase inhibitor may be eflornithine, in particular eflornithine hydrochloride. Eflornithine, and salts thereof, corresponding methods of synthesis, and methods of testing activity are described in U.S. Pat. No. 4,413,141, which is incorporated herein by reference in its entirety. Eflornithine hydrochloride also known as VANIQA® is (±)-2-(difluoromethyl) omithine monohydrochloride monohydrate, with the empirical formula C₆H₁₂F₂N₂O₂·HCl·H₂O, a molecular weight of 236.65 and the following structural formula, Formula (I):

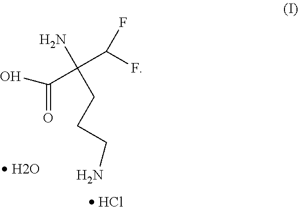 [SMILES: "· H2O" ; NCCCC(N)(C(=O)O)C(F)F ; "· HCl"]

VANIQA® is described by U.S. Pat. Nos. 5,648,394 and 4,720,489, each of which is incorporated herein by reference in their entireties. Anhydrous eflornithine hydrochloride has an empirical formula C₆H₁₂F₂N₂O₂·HCl and a molecular weight of 218.65. Other ingredients included in VANIQA® may be ceteareth-20, cetearyl alcohol, dimethicone, glyceryl stearate, methylparaben, mineral oil, PEG-100 stearate, phenoxyethanol, propylparaben, stearyl alcohol, and water. Eflornithine hydrochloride irreversibly inhibits in skin the activity of the enzyme omithine decarboxylase (ODC). ODC is necessary in the synthesis of polyamines.

Additional suitable compounds that may be used for inhibiting pigmentation of hair or skin may include the following: prostaglandin F2a (FP) receptor antagonists (U.S. Ser. No. 10/159,634B2, US2007/0004620A1, U.S. Ser. No. 10/259,795B2, U.S. Ser. No. 11/149,018B2; such as OBE002 ([(S)-3-(biphenyl-4-sulfonyl)-thiazolidine-2-carboxylic acid [(S)-1-(4-fluoro-phenyl)-3-hydroxy-propyl]-amide], OBE022 ([(S)-2-amino-3-methyl-butyric acid (S)-3-{[(S)-3-(biphenyl-4-sulfonyl)-thiazolidine-2-carbonyl]-amino}-3-(4-fluoro-phenyl)-propyl ester], and BAY-6672 ((4R)-5-[(6-bromo-3-methyl-2-pyrrolidin-1-ylquinoline-4-carbonyl)amino]-4-(2-chlorophenyl)pentanoic acid)); a prostaglandin D2 receptor, DP-2 agonist (WO2013142295A1); prostaglandin synthetase inhibitors; a Wnt Inhibitor (US20120165270A1); advanced glycation end products (AGE's) and compounds such as lysine (WO2010063678A2, WO2010063673A2); extracts of tetraselmis species (US20100143267A1); fibroblast growth factor (FGF)18 (WO2008102782A1); FGF5 (US20160009797A1); cytotoxic lectin (US20080145330A1); trypsin and other enzymes (US20070269418A1); extract of Juniperus genus and/or malt extract (U.S. Pat. Nos. 6,375,948 and 7,211,278); hair growth-inhibiting active substances (WO06125582A1); extract of ginger root (US20060099280A1); toxalbumins such as ricin, abrin, or modeccin and the like (US20060034952A1); inhibitors of cysteine pathway enzymes (WO9524885A1); inhibitors of nitric oxide synthetase (WO9524884A1); omithine amino transferase inhibitors (WO9524181A1); cyclooxygenase inhibitors such as NSAIDs (WO9427586A1); 5-lipoxygenase inhibitors (WO9427563A1) substances such as substituted guanidines or amidines (WO8808295A1); heparanase inhibitors or antisense oligonucleotides targeted to heparanase (WO2004006949A1); nicotinamide (WO2016102969A1); inhibitor of phosphofructokinase (U.S. Pat. No. 5,824,665); DNA polymerase inhibitor (US20100137341A1); heat shock protein inhibitor (US20090182031A1); methyl palmoxirate (U.S. Pat. No. 7,160,921); nordihydroguaiaretic acid (U.S. Pat. No. 6,414,017); NK1 receptor antagonists; platelet activating factor receptor antagonists (U.S. Pat. No. 6,093,748); and aminobenzophenones (U.S. Pat. No. 3,426,137). Each of the foregoing references is incorporated herein by reference in their entireties.

The FP receptor antagonist may be a compound of Formula (II), or a pharmaceutically acceptable salt thereof,

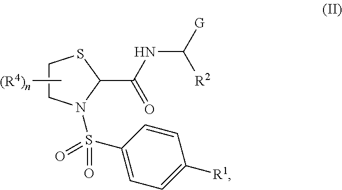

wherein G, R1, R2, R4, and n are as described below.
G may be

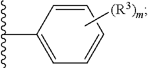

R¹ may be tert-butyl or phenyl, wherein the phenyl is optionally substituted with 1-3 R^{1a};
R^{1a}, at each occurrence, may be independently C₁₋₄alkyl, C₁₋₄fluoroalkyl, halogen, cyano, —OC₁₋₄alkyl, or —OC₁₋₄fluoroalkyl;
R² may be —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a};
R^{2a} may be hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl;
R³, at each occurrence, may be independently C₁₋₄alkyl, C₁₋₄fluoroalkyl, halogen, cyano, —OC₁₋₄alkyl, or —OC₁₋₄fluoroalkyl;
R⁴, at each occurrence, may be independently C₁₋₄alkyl or C₁₋₄fluoroalkyl;
m may be 0, 1, 2, 3, 4, or 5; and
n may be 0, 1, 2, 3, or 4.

In an embodiment, R¹ may be phenyl.

In an embodiment, R² may be —C₁₋₄alkylene-OH.

In an embodiment, R² may be —CH₂CH₂OH.

In an embodiment, R² may be —C₁₋₄alkylene-OC(O)CH(NH₂)R^{2a}, such as (S)—C₁₋₄alkylene-OC(O)CH(NH₂)R^{2a} or (S)—C₁₋₄alkylene-OC(O)CH(NH₂)R^{2a}. Preferably, —C₁₋₄alkylene-OC(O)CH(NH₂)R^{2a} may be (S)—C₁₋₇alkylene-OC(O)CH(NH₂)R^{2a}. R² may be —CH₂CH₂—OC(O)CH(NH₂)R^{2a}, such as (S)—CH₂CH₂—OC(O)CH(NH₂)R^{2a} or (R)—CH₂CH₂—OC(O)CH(NH₂)R^{2a}. Preferably, —CH₂CH₂—OC(O)CH(NH₂)R^{2a} may be (S)—CH₂CH₂—OC(O)CH(NH₂)R^{2a}. R² may be —C₁₋₄alkylene-OC(O)CH(NH₂)CH(CH₃)₂, such as (S)—C₁₋₄alkylene-OC(O)CH(NH₂)CH(CH₃)₂ or (R)—C₁₋₄alkylene-OC(O)CH(NH₂)CH(CH₃)₂. Preferably, —C₁₋₄alkylene-OC(O)CH(NH₂)CH(CH₃)₂ may be (S)—C₁₋₄alkylene-OC(O)CH(NH₂)CH(CH₃)₂. R² may be —CH₂CH₂—OC(O)CH(NH₂)CH(CH₃)₂, such as (S)—CH₂CH₂—OC(O)CH(NH₂)CH(CH₃)₂ or (R)—CH₂CH₂—OC(O)CH(NH₂)CH(CH₃)₂. Preferably, —CH₂CH₂—OC(O)CH(NH₂)CH(CH₃)₂ may be (S)—CH₂CH₂—OC(O)CH(NH₂)CH(CH₃)₂. Preferred pharmaceutically acceptable salts of the primary amino group include the hydrochloride, mesylate, hydrosulfate, sulfate, dihydrophosphate, citrate, edisylate, and fumarate.

In an embodiment, R³ may be fluoro, chloro, CH₃, or OCH₃; and m may be 0, 1, or 2.

In an embodiment, G may be

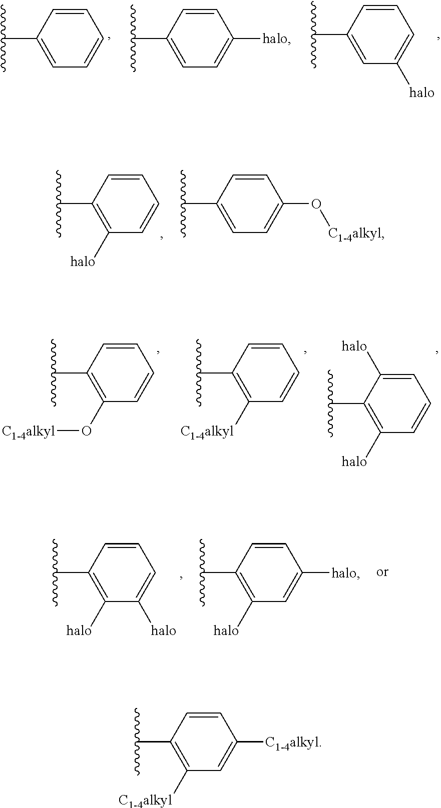

In an embodiment, G may be

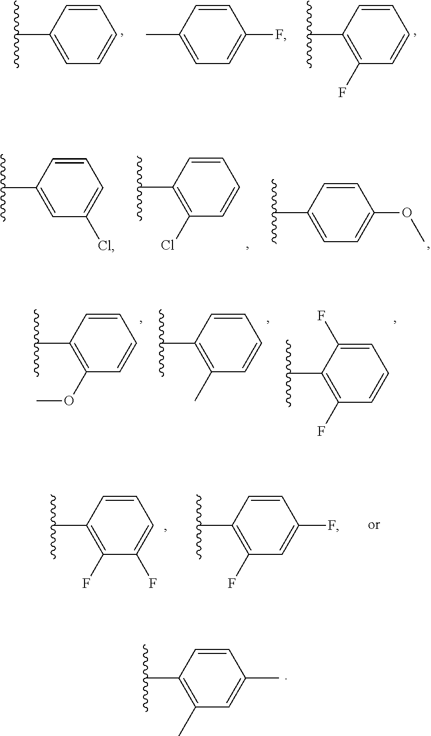

In an embodiment, n may be 0.

The compound of formula (II) may have formula (II-A)

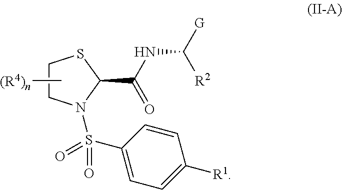

The compound of formula (II) may be

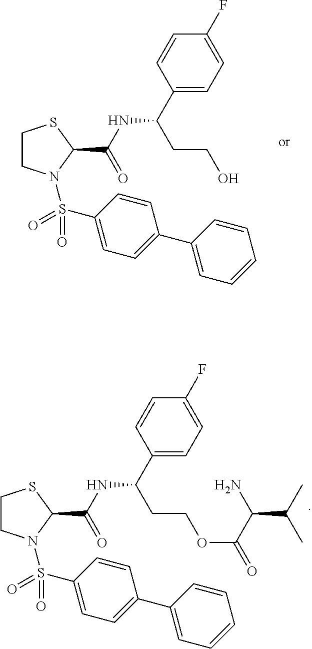 [SMILES: O=C(N[C@@H](CCO)c1ccc(F)cc1)[C@@H]1SCCN1S(=O)(=O)c1ccc(-c2ccccc2)cc1 ; CC(C)[C@H](N)C(=O)OCC[C@H](NC(=O)[C@@H]1SCCN1S(=O)(=O)c1ccc(-c2ccccc2)cc1)c1ccc(F)cc1]

The compounds of formula (II), and salts thereof, corresponding methods of synthesis, and methods of testing for FP receptor antagonist activity are described in U.S. Pat. Nos. 8,415,480, 9,447,055, 9,834,528, and 10,259,795 which are incorporated herein by reference.

The FP receptor antagonist may be a compound of Formula (III), or a pharmaceutically acceptable salt thereof,

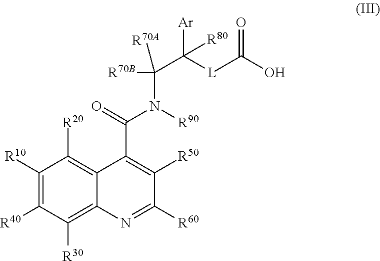

wherein R¹⁰, R²⁰, R³⁰, R⁴⁰, R⁵⁰, R⁶⁰, R^{70A}, R^{70B}, R⁸⁰, R⁹⁰, and Ar are as described below.

Ar may be phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₃₋₄cycloalkyl, —OC₁₋₂alkyl, —SC₁₋₂alkyl, C₁₋₄alkyl substituted by 1-3 fluoro, C₃₋₄cycloalkyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy;
L may be a bond or —X—(CR^{a}R^{b})—;
X may be a bond, CH₂, O, S, S(O), S(O)₂, or N(R);
R may be hydrogen or CH₃;
R^{a} and R^{b} may be independently hydrogen, fluoro, or CH₃; or R^{a} and R^{b} together with the carbon atom to which they attach form a cyclopropyl;
k may be 1, 2, 3, or 4;
R¹⁰ may be halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ may be independently hydrogen, halogen, CH₃, CH₂F, CHF₂, or CF₃;
R⁵⁰ may be halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ may be —NR^{c}R^{d};
R^{c} may be hydrogen or C₁₋₃alkyl;
R^{d} may be C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach may form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings may be optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} may be independently hydrogen or CH₃, or R^{70A} and R^{70B} together with the carbon atom to which they attach form a cyclopropyl;
R⁸⁰ may be hydrogen, fluoro, CH₃, CF₃, CH₂CH₃, or OH; and
R⁹⁰ may be hydrogen or CH₃.

In an embodiment, Ar may be phenyl, wherein the phenyl may be unsubstituted or substituted with 1-3 substituents independently selected from the group consisting of fluoro, chloro, CH₃, CF₃, OCH₃, OCHF₂, and OCF₃;
In an embodiment, L may be —CH₂CH₂—;
In an embodiment, R¹⁰ may be bromo;
In an embodiment, R⁵⁰ may be CH₃;
In an embodiment, R⁶⁰ may be

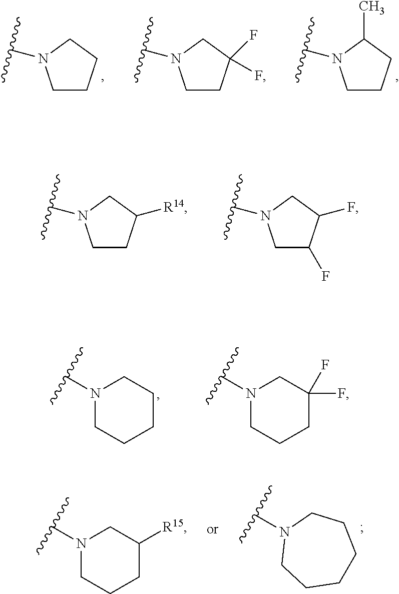

In an embodiment, R¹⁴ may be fluoro or CH₃; and
In an embodiment, R¹⁵ may be fluoro, CH₃, or CH₂CH₃.

The compound of formula (III) may be

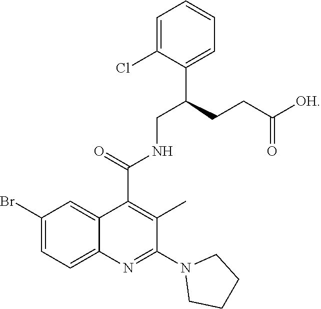 [SMILES: Cc1c(N2CCCC2)nc2ccc(Br)cc2c1C(=O)NC[C@H](CCC(=O)O)c1ccccc1Cl]

The compounds of formula (III), and salts thereof, corresponding methods of synthesis, and methods of testing for FP receptor antagonist activity are described in US2020/0157073, which is incorporated herein by reference.

Additional suitable compounds that may be used as secondary agents or in combination with a compound for inhibiting pigmentation of hair or skin may include known skin and/or hair lightening agents, known hirsutism treatment agents, and/or known preventatives of chemotherapy- or radiation-induced alopecia or hair loss.

Suitable known skin lightening agents may include the following: acetylcholinesterase inhibitors (WO2010066639A2); terpenes (WO2010015487A3); Phyllanthus emblica fruit extract, Bellis perennis flower extract, and licorice root extract (WO2010059140A1); 4-methyl-7-hydroxycoumarin derived resorcinol derivatives (U.S. Pat. No. 7,723,537); 3-phenyl-chromen-4-one derivatives (WO2010009578A1); 3-dithiane resorcinol derivatives (US20090175812A1); goya or its extract and pine bark extracts (US20080268079A1); hydroquinone or its derivatives such as retinoids, azelaic acid, and N-acylbenzothiazolone compounds (U.S. Pat. No. 7,270,805); niacinamide, cucumber and lemon extracts, 2-imino-imidazolidin-4-one derivatives and phenyl derivatives (c.f., US20060216254A1); vasoconstrictors (WO06031555A2); Emblicanin A, Emblicanin B, Pedunculagin, Punigluconin, and flavonoids (U.S. Pat. No. 6,969,509); 4-substituted-7-hydroxycoumarin derived compounds (WO05085169A1); phenyl glycine derivatives (U.S. Pat. No. 6,824,786); skin lightening proteins, sulfobenzoic acids, and their derivatives (WO03051325A1); dihydroxybenzene derivatives (US20030072726A1); N-acetyl-aldosamines or N-acetylamino acids (U.S. Pat. No. 6,524,593, reissued in 2010 as USRE41278); and ellagic acid-based compounds, Kojic acid, salicylic acid, and the deoxyarbutins (U.S. Pat. No. 6,068,834, which is incorporated herein by reference in its entirety). In certain embodiments, the skin lightening agent is deoxyArbutin or GirLite®. Each of the foregoing references is incorporated herein by reference in their entireties.

Suitable known hirsutism treatment agents may include the following: botulinum toxin (U.S. Pat. No. 7,754,253); spironolactone (WO9936030A3, WO8700427A1); 2-phenyl-benzothiophene derivatives (U.S. Pat. No. 5,686,468); cyproterone acetate, flutamide, bicalutamide, and inhibitors of 5-alpha reductase such as finasteride dutasteride (U.S. Pat. Nos. 7,744,935, 7,737,288, 7,727,980); N,N-diethyl-4-methyl-3-oxo-4-aza-5α-androstane-17β-carboxamide (4-MA, WO9906050A1); PTHR1 receptor ligands (WO2010053548A2); ketaconazole; estrogen receptor modulators such as oral contraceptives (U.S. Pat. No. 5,770,226); progesterone; estrogen (US20070105827); RU58841; neuropeptide Y receptor antagonists; thiazolidinedione derivatives such as rosiglitazone or pioglitazone (U.S. Pat. No. 5,972,944); biguanide (metformin) derivatives; cyoctol [6-(5-methoxy-1-heptyl)-bicyclo (3,3,0)octan-3-one]; botanicals including extracts of Serenoa repens (WO9833472A1); Epilobium species; Cucurbita pepo (U.S. Pat. No. 7,595,346); Urtica dioica; Calluna vulgaris; Populus species; Barosma species; and physical means of hair removal such as laser, electrolysis, and depilatory compounds. Each of the foregoing references is incorporated herein by reference in their entireties.

Suitable known preventatives of chemotherapy- or radiation-induced alopecia or hair loss may include the following: 4-((cyanoimino((1,2,2-trimethylpropyl) amino)methy)amino) benzonitrile (U.S. Pat. No. 6,458,835); growth factors including keratinocyte growth factor, epidermal growth factor, and fibroblast growth factor; prostaglandins including PGE2 and Misoprostol (U.S. Pat. Nos. 7,407,987, 7,388,029); ImuVert; AS101; IL-1; cyclin dependent kinases; p53 inhibitors; caspase-3 inhibitors; acylated amino acids including N-acyl cysteine (US20060211659A9); nuclear hormone receptor ligands such as parathyroid hormone antagonist; vitamin and vitamin derivatives such as alpha-tocopherol; M50054; immunosuppressant agents especially cyclosporine; angiotensin receptor blockers (US20060135422A1); and oral or topical minoxidil. Each of the foregoing references is incorporated herein by reference in their entireties.

### 3. PHARMACEUTICAL COMPOSITIONS

A compound as described herein may be incorporated into pharmaceutical compositions suitable for administration to a subject, such as a patient. The pharmaceutical composition may be prepared for administration to a subject. Such pharmaceutical compositions can be administered in dosages and by techniques well known to those skilled in the medical and pharmaceutical arts taking into consideration such factors as the age, sex, weight, and condition of the particular subject, and the route of administration. The compositions may be for topical or oral administration. In an embodiment, a topical composition may be prepared by methods as described in U.S. Pat. No. 5,648,394.

A compound as described herein is an active ingredient formulated into a composition, such as a pharmaceutical or cosmetic composition, administered for treatment or prophylaxis of a condition, including, for example, inhibition of pigmentation of hair and/or skin in mammals, and/or unwanted dark shade of hair. Standard pharmaceutical formulation techniques are used, such as those disclosed in Remington's Pharmaceutical Sciences, Mack Publishing Company, Easton, PA. (1990).

The composition may further comprise a carrier. “Carrier” means one or more compatible substances that are suitable for administration to a mammal. Carrier includes solid or liquid fillers, diluents, hydrotopes, surface-active agents, and encapsulating substances. “Compatible” means that the components of the composition are capable of being commingled with a compound as described herein, and with each other, in a manner such that there is no interaction which would substantially reduce the efficacy of the composition under ordinary use situations. Carriers must be of sufficiently high purity and sufficiently low toxicity to render them suitable for administration to the mammal being treated. The carrier can be inert, or it can possess pharmaceutical benefits, cosmetic benefits, or both.

The choice of carrier depends on the route by which a compound as described herein will be administered and the form of the composition. The composition may be in a variety of forms, suitable, for example, for systemic administration (e.g., oral, rectal, nasal, sublingual, buccal, or parenteral) or topical administration (e.g., local application on the skin, ocular, liposome delivery systems, or iontophoresis). Topical and/or oral administration is preferred.

Carriers for systemic administration typically comprise one or more ingredients selected from the group consisting of diluents, lubricants, binders, disintegrants, colorants, flavors, sweeteners, antioxidants, preservatives, glidants, solvents, suspending agents, wetting agents, surfactants, combinations thereof, and others.

Suitable diluents include sugars such as glucose, lactose, dextrose, and sucrose; polyols such as propylene glycol; calcium carbonate; sodium carbonate; cellulose; glycerin; mannitol; and sorbitol.

Suitable lubricants are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma.

Suitable binders include polyvinylpyrolidone; magnesium aluminum silicate; starches such as corn starch and potato starch; gelatin; tragacanth; and cellulose and its derivatives, such as sodium carboxymethylcellulose, ethyl cellulose, methylcellulose, and sodium carboxymethylcellulose.

Suitable disintegrants include starches, agar, alginic acid and the sodium salt thereof, effervescent mixtures, and croscarmellose.

A colorant may be an FD&C dye.

A flavor may be menthol, peppermint, and/or fruit flavors.

A sweetener may be aspartame and/or saccharin.

An antioxidant may be BHA, BHT, and/or vitamin E.

A preservative may be methyl paraben and/or sodium benzoate.

A glidant may be silicon dioxide.

A solvent may be water, isotonic saline, ethyl oleate, alcohols such as ethanol, and/or phosphate buffer solutions.

Suitable suspending agents include cellulose and its derivatives, such as methyl cellulose and sodium carboxymethyl cellulose; Avicel® RC-591 from FMC Corporation of Philadelphia, Pennsylvania; tragacanth and sodium alginate.

A wetting agent may be lecithin, polysorbate 80, and/or sodium lauryl sulfate.

A surfactant may be the TWEENS® from Atlas Powder Company of Wilmington, Delaware.

Compositions for parenteral administration typically comprise 0.1% to 10% of a compound as described herein and 90% to 99.9% of a carrier comprising a diluent, a lubricant, a binder, and a solvent.

Compositions for oral administration can have various dosage forms. For example, solid forms include tablets, capsules, granules, and bulk powders. These oral dosage forms comprise a safe and effective amount, usually at least 5%, and preferably from 25% to 50%, of a compound as described herein. The oral dosage compositions further comprise 50% to 95% of a carrier, preferably 50% to 75%.

Tablets can be compressed, tablet triturates, enteric-coated, sugar-coated, film-coated, or multiple-compressed. Tablets typically comprise a compound as described herein, and a carrier comprising ingredients selected from the group consisting of diluents, lubricants, binders, disintegrants, colorants, flavors, sweeteners, glidants, and combinations thereof. Preferred diluents include calcium carbonate, sodium carbonate, mannitol, lactose and cellulose. Preferred binders include starch, gelatin, and sucrose. Preferred disintegrants include starch, alginic acid, and croscarmellose. Preferred lubricants include magnesium stearate, stearic acid, and talc. Preferred colorants are the FD&C dyes, which can be added for appearance. Chewable tablets preferably contain sweeteners such as aspartame and saccharin, or flavors such as menthol, peppermint, and fruit flavors.

Capsules (including time release and sustained release formulations) typically comprise a compound as described herein, and a carrier comprising one or more diluents disclosed above in a capsule comprising gelatin. Granules typically comprise a compound as described herein, and preferably further comprise glidants such as silicon dioxide to improve flow characteristics.

The selection of ingredients in the carrier for oral compositions depends on secondary considerations like taste, cost, and shelf stability. One skilled in the art would know how to select appropriate ingredients without undue experimentation.

The solid compositions may also be coated by conventional methods, typically with pH or time-dependent coatings, such that a compound as described herein is released in the gastrointestinal tract in the vicinity of the desired application, or at various times to extend the desired action. The coatings typically comprise one or more components selected from the group consisting of cellulose acetate phthalate, polyvinylacetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise a compound as described herein and a carrier comprising ingredients selected from the group consisting of: diluents, colorants, flavors, sweeteners, preservatives, solvents, suspending agents, and surfactants. Peroral liquid compositions preferably comprise one or more ingredients selected from the group consisting of colorants, flavors, and sweeteners.

Other compositions useful for attaining systemic delivery of the subject compounds include sublingual, buccal and nasal dosage forms. Such compositions typically comprise one or more of soluble filler substances such as diluents including sucrose, sorbitol and mannitol; and binders such as acacia, microcrystalline cellulose, carboxymethyl cellulose, and hydroxypropyl methyl cellulose. Such compositions may further comprise lubricants, colorants, flavors, sweeteners, antioxidants, and glidants.

The compositions may further comprise an optional activity enhancer. The activity enhancers are exemplified by compounds that work marginally, if at all by itself, but can help the activity of the compounds as described herein.

A penetration enhancer can be added to all of the compositions for systemic administration except compositions for oral administration. The amount of penetration enhancer, when present in the composition, may typically be 1% to 5%. Examples of penetration enhancers include 2-methyl propan-2-ol, propan-2-ol, ethyl-2-hydroxypropanoate, hexan-2,5-diol, POE(2) ethyl ether, di(2-hydroxypropyl) ether, pentan-2,4-diol, acetone, POE(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, POE ester of oleyl alcohol, oleyl alcohol, lauryl alcohol, dioctyl adipate, dicapryl adipate, di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, iso-propyl palmitate, ethyl laurate, 2-ethyl-hexyl pelargonate, iso-propyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hydroxyoctanoic acid, dimethyl sulphoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacycloheptan-2-one, and combinations thereof.

In certain embodiments, the compounds as described herein are topically administered. Topical compositions that can be applied locally to the skin may be in any form including intralesional injections, solutions, oils, creams, ointments, gels, lotions, shampoos, leave-on and rinse-out hair conditioners, milks, cleansers, moisturizers, mousses, sprays, foam, skin patches, and the like. The compound or composition may be administered to a subject with an agent that increases absorption of the compound such as a low-level laser. Topical compositions may comprise: a compound as described herein and a carrier. The carrier of the topical composition may aid penetration of the compounds as described herein into the skin to reach the environment where the compounds are needed. The carrier may further comprise one or more optional components described above.

The exact amounts of each component in the topical composition depend on various factors. The amount of the compound depends on the binding affinity (IC50) of the compound selected. The amount of the compound added to the topical composition is:

$${IC}_{50} \times {10}^{- 1} \geq \% of a compound described herein \geq {IC}_{50} \times {10}^{- 5} ,$$

where IC₅₀ is expressed in nanomolar. For example, if the binding affinity of the compound is 1 nM, the amount of the compound will be 0.00001% to 0.1%. If the binding affinity of the compound is 10 nM, the amount of the compound will be 0.0001% to 0.1%. The amount and dosage of a compound as described herein are critical. If the amount of the compound is outside the ranges specified above (i.e., either higher or lower), efficacy of the treatment will be reduced.

The carrier may comprise a single component or a combination of two or more components. Typical carriers in the topical compositions may include water, alcohols, aloe vera gel, allantoin, glycerin, vitamin A and E oils, mineral oil, propylene glycol, PPG-2 myristyl propionate, dimethyl isosorbide, combinations thereof, and the like. Preferred carriers include alcohols, mineral oil, and water.

The carrier of the topical composition may further comprise one or more ingredients selected from the group consisting of emollients, propellants, solvents, humectants, thickeners, powders, and fragrances.

The amount of an emollient in the topical composition may be 5% to 95%. Suitable emollients include stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, polydimethylsiloxane, di-n-butyl sebacate, iso-propyl myristate, Iso-propyl palmitate, Iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, Isostearic acid, palmitic acid, isopropyl linoleate, lauryl lactate, myristyl lactate, decyl oleate, myristyl myristate, and combinations thereof. Preferred emollients include stearyl alcohol and polydimethylsiloxane.

The amount of a propellant in the topical composition may be 5% to 95%. Suitable propellants include propane, butane, iso-butane, dimethyl ether, carbon dioxide, nitrous oxide, and combinations thereof.

The amount of a solvent in the topical composition may be 5% to 95%. Suitable solvents include water, ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran, and combinations thereof. Preferred solvents include ethyl alcohol.

The amount of a humectant in the topical composition may be 5% to 95%. Suitable humectants include glycerin, sorbitol, sodium 2-pyrrolidone-5-carboxylate, soluble collagen, dibutyl phthalate, gelatin, and combinations thereof. Preferred humectants include glycerin.

The amount of a thickener in the topical composition may be 0% to 95%.

The amount of a powder in the topical composition may be 0% to 95%. Suitable powders include chalk, talc, fullers earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

The amount of a fragrance in the topical composition may be 0.001% to 0.5%, preferably 0.001% to 0.1%.

Any activity enhancers may be added to the topical compositions. In certain embodiments, the topical composition may comprise 0.01% to 15% of at least one of the activity enhancers. More preferably, the composition comprises 0.1% to 10%, and most preferably 0.5% to 5% of at least one of the activity enhancers. In certain embodiments, the topical composition comprises 1% to 5% of an activity enhancer.

In certain embodiments, pharmaceutical compositions may further comprise additional active agents including, but not limited to, sunscreens and sunblocks, anti-oxidants/radical scavengers, topical steroids, and retinoids.

In an alternative embodiment of the invention, pharmaceutical compositions for topical administration are prepared by conventional methods. Pharmaceutical compositions for topical administration typically comprise a compound as described herein, a carrier, such as purified water, and one or more ingredients selected from the group consisting of sugars such as dextrans, particularly dextran 70, cellulose or a derivative thereof, a salt, disodium EDTA (Edetate disodium), and a pH adjusting additive.

Examples of cellulose derivatives suitable for use in the pharmaceutical composition for topical administration include sodium carboxymethyl cellulose, ethyl cellulose, methyl cellulose, and hydroxypropylmethylcellulose. Hydroxypropylmethylcellulose is preferred.

Examples of salts suitable for use in the for use in the pharmaceutical composition for topical administration include sodium chloride, potassium chloride, and combinations thereof.

Examples of pH adjusting additives include HCl or NaOH in amounts sufficient to adjust the pH of the pharmaceutical composition for topical administration to 5.2-7.5.

The compounds as described herein may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. A preferred formulation for topical delivery of the present compounds uses liposomes as described in Dowton at al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Walach and Philippot, “New Type of Lipid Vesicle: Novasome”, Liposome Technology, Vol. 1, pp. 141-156 (1993); Wallach, U.S. Pat. No. 4,911,928, assigned to Micro-Pak, Inc., Issued Mar. 27, 1990; and Weiner et al., U.S. Pat. No. 5,834,014, assigned to The University of Michigan and Micro-Pak, Inc., issued Nov. 10, 1998 (with respect to Weiner et al., with a compound as described herein administered in lieu of, or in addition to, minoxidil).

The compounds as described herein may also be administered by iontophoresis. See, e.g., internet site www.unipr.it/arpa/dipfarm/erasmus/erasm14.html; Banga et al., “Hydrogel-based Iontotherapeutic Delivery Devices for Transdermal Delivery of Peptide/Protein Drugs”, Pharm. Res., Vol. 10 (5), pp. 697-702 (1993); Ferry, “Theoretical Model of Iontophoresis Utilized in Transdermal Drug Delivery”, Pharmaceutical Acta Helvetiae, Vol 70, pp. 279-287 (1995); Gangarosa at al., “Modern Iontophoresis for Local Drug Delivery”, Int. J. Pharm, Vol. 123, pp. 159-171 (1995); Green et al., “Iontophoretic Delivery of a Series of Tripeptides Across the Skin in vitro”, Pharm. Res., Vol 8, pp. 1121-1127 (1991); Jadoul et al., “Quantification and Localization of Fentanyl and TRH Delivered by Iontophoresis in the Skin”, Int. J. Pharm., Vol. 120, pp. 221-8 (1995); O'Brien et al., “An Updated Review of its Antiviral Activity, Pharmacokinetic Properties and Therapeutic Efficacy”, Drugs, Vol. 37, pp. 233-309 (1989); Parry et al., “Acyclovir Bioavailability in Human Skin”, J. Invest. Dermatol., Vol. 98 (6), pp. 856-63 (1992); Santi et al., “Drug Reservoir Composition and Transport of Salmon Calcitonin in Transdermal Iontophoresis”, Pharm. Res., Vol 14 (1), pp. 63-66 (1997); Santi et al., “Reverse Iontophoresis—Parameters Determining Electroosmotic Flow: I. pH and Ionic Strength”, J. Control. Release, Vol. 38, pp. 159-165 (1996); Santi et al., “Reverse Iontophoresis—Parameters Determining Electroosmotic Flow: II. Electrode Chamber Formulation”, J. Control. Release, Vol. 42, pp. 29-36 (1996); Rao et al., “Reverse Iontophoresis: Noninvasive Glucose Monitoring in vivo in Humans”, Pharm. Res., Vol. 12 (12), pp. 1869-1873 (1995); Thysman et al., “Human Calcitonin Delivery in Rats by Iontophoresis”, J. Pharm. Pharmacol., Vol. 46, pp. 725-730 (1994); and Volpato et al., “Iontophoresis Enhances the Transport of Acyclovir through Nude Mouse Skin by Electrorepulsion and Electroosmosis”, Pharm. Res., Vol. 12 (11), pp. 1623-1627 (1995).

### 4. METHODS

This disclosure further relates to a method for inhibition of pigmentation of hair and/or skin in mammals, and/or unwanted dark shade of hair in mammals. The method comprises administering to a mammal (preferably a human) a compound as described herein. For example, a mammal diagnosed with melasma, chloasma, postinflammatory hyperpigmentation, lentigines, or hyperpigmentation can be treated by the methods of this disclosure. The ability to lighten the overall pigment of a mammal's skin can be treated by the methods of this disclosure. Preferably, a topical composition as described herein may be administered to the mammal. A compound or composition as described herein may be administered orally or applied topically to the scalp, eyebrows, beard, moustache, eyelashes, body hair, skin of any part of the body, or a combination thereof. The compound or composition may be administered for at least 1, 2, 3, 4, 5, 6, 7, 8, 9, 10, 11, 12, or 13 days. The topical composition is typically administered from once per day up to four times per day. In general, 6-10 weeks is sufficient to observe a noticeable decrease in pigmentation.

The dosage of a compound as described herein administered to a subject depends on the method of administration. For systemic administration, (e.g., oral, rectal, nasal, sublingual, buccal, or parenteral), typically, 0.1 mg to 300 mg, preferably 0.5 mg to 100 mg, more preferably 0.1 mg to 10 mg, of a compound described above may be administered per day. These dosage ranges are merely exemplary, and daily administration can be adjusted depending on various factors. The specific dosage of the compound to be administered, as well as the duration of treatment, and whether the treatment is topical or systemic are interdependent. The dosage and treatment regimen will also depend upon such factors as the specific compound used, the treatment indication, the efficacy of the compound, the personal attributes of the subject (such as, for example, weight, age, sex, and medical condition of the subject), compliance with the treatment regimen, and the presence and severity of any side effects of the treatment.

For topical administration (e.g., local application on the skin, ocular, liposome delivery systems, intralesional injections, shampoos, lotions, solutions, with agents such as low-level laser that increase absorption, or iontophoresis), a compound as described herein or topical composition may typically be administered from once per day up to six times per day. The compound or composition may be administered once, twice, three, four, five, or six times daily. The compound or composition may be administered daily to weekly. About 0.0001 g to about 25 g of the compound of composition may be administered to a subject per day. About 0.5 g to about 1.0 g of the compound or composition may be administered to a subject per day.

A compound as described herein may be included in kits comprising a compound as described herein, a systemic or topical composition described above, or both; and information, instructions, or both that use of the kit will provide treatment for conditions including, for example, melasma, chloasma, postinflammatory hyperpigmentation, lentigines, or hyperpigmentation in mammals (particularly humans). The information and instructions may be in the form of words, pictures, or both, and the like. In addition, or in the alternative, the kit may comprise a composition as described herein, a composition as described herein, or both; and information, instructions, or both, regarding methods of application of the compound or composition, preferably with the benefit of inhibiting pigmentation of hair and/or skin and/or unwanted dark shade of hair in mammals.

In all of the foregoing compositions, and for all routes of administration, a compound as described herein can be used alone or in combinations of two or more compounds described herein. The compositions may further comprise additional drugs or excipients as appropriate for the indication.

### 5. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples. The following examples are prophetic.

### Example 1

Compositions for topical administration may be made, comprising:

| Component | Wt. % |
|---|---|
| A compound as described | 1-20 |
| herein | |
| Water | 78-87 |
| Glyceryl Stearate | 2.8-4.8 |
| PEG-100 Stearate | 2.7-4.7 |
| Cetearyl Alcohol | 1.9-3.3 |
| Ceteareth-20 | 1.6-2.7 |
| Mineral Oil | 1.7-2.7 |
| Stearyl Alcohol | 1.0-2.0 |
| Dimethicone | 0.3-1.0 |
| Citric Acid | 0.0-0.5 |
| Sodium Hydroxide | Quantum |
| | Sufficient |
| | (q.s.) |

| Component | Wt. % |
|---|---|
| A compound as described herein | 3-8 |
| Polyol (e.g., propylene glycol, dipropylene glycol, hexylene | 30-80 |
| glycol, 1,3-butylene glycol, liquid polyethylene glycols, such | |
| as polyethylene glycol 200 (PEG-200) and polyethylene | |
| glycol 400 (PEG-400), and glycerol) | |
| Alcohol (e.g., ethanol, propanol, and butanol) | 10-50 |
| Non-Carbomeric Polymer (e.g., Carbopols, such as | 0.01-50 |
| Carbopol ® 934P, Carbopol ® 940, Carbopol ® 980, | |
| Carbopol ® 981, and Carbopol ® Ultrez ™ 10) | |
| Neutralizing Agent (e.g., 2-amino-2-methyl-1-propanol, | 0-3 |
| diisopropanolamine, triisopropanolamine and | |
| tetrahydroxypropyl ethylenediamine, and mixtures thereof) | |
| Water | q.s. |

According to the methods described herein, a subject may be treated with the above composition. Specifically, for 16 weeks, one of the above compositions will be administered daily, topically to the subject.

### Example 2

A composition for topical administration may be made according to the method of Dowton t al., Influence of Liposomal Composition on Topical Delivery of Encapsulated Cyclosporin A: I. An in vitro Study Using Hairless Mouse Skin, S.T.P. Pharma Sciences, Vol. 3, pp. 404-407 (1993), using a compound as described herein in lieu of cyclosporin A and using the NOVASOME® 1 (available from Micro-Pak, Inc. of Wilmington, Delaware) for the non-ionic liposomal formulation. According to the methods described herein, a subject may be treated with the above composition. Specifically, for 16 weeks, the above composition will be administered topically to the subject.

### Example 3

Shampoos or body washes may be made, comprising:

| Component | E | F | G | H |
|---|---|---|---|---|
| Ammonium Lauryl Sulfate | 11.5% | 11.5% | 9.5% | 7.5% |
| Ammonium Laureth Sulfate | 4% | 3% | 2% | 2% |
| Cocamide MEA | 2% | 2% | 2% | 2% |
| Ethylene Glycol Distearate | 2% | 2% | 2% | 2% |
| Cetyl Alcohol | 2% | 2% | 2% | 2% |
| Stearyl Alcohol | 1.2% | 1.2% | 1.2% | 1.2% |
| Glycerin | 1% | 1% | 1% | 1% |
| Polyquaternium 10 | 0.5% | 0.25% | — | — |
| Polyquaternium 24 | — | — | 0.5% | 0.25% |
| Sodium Chloride | 0.1% | 0.1% | 0.1% | 0.1% |
| Sucrose Polyesters of | 3% | 3% | — | — |
| Cottonate Fatty Acid | | | | |
| Sucrose Polyesters of | 2% | 3% | — | — |
| Behenate Fatty Acid | | | | |
| Polydimethyl Siloxane | — | — | 3% | 2% |
| Cocaminopropyl Betaine | — | 1% | 3% | 3% |
| Lauryl Dimethyl Amine Oxide | 1.5% | 1.5% | 1.5% | 1.5% |
| Decyl Polyglucose | — | — | 1% | 1% |
| DMDM Hydantoin | 0.15% | 0.15% | 0.15% | 0.15% |
| A compound as described herein | 1% | 0.5% | 3% | 2% |
| Phenoxyethanol | 0.5% | 0.5% | 0.5% | 0.5% |
| Fragrance | 0.5% | 0.5% | 0.5% | 0.5% |
| Water | q.s. | q.s. | q.s. | q.s. |

According to the methods described herein, a subject may be treated with the above composition. Specifically, for 12 weeks, a shampoo or body-wash selected from the ones described above will be used daily by the subject.

### Example 4

Shampoos and/or body washes may be made according to Example 3. A human subject who may have melasma, chloasma, postinflammatory hyperpigmentation, lentigines, or hyperpigmentation, or be in need of inhibiting pigmentation of hair and/or skin and/or unwanted dark shade of hair and/or skin may be treated by a method as described herein. Specifically, for days to several weeks, a shampoo or body wash selected from the ones described above may be used daily by the subject by applying to the scalp or body. Solution may also be applied to the face, eyebrows, or eyelashes. The treatment will reduce pigmentation of the skin and/or hair.

### Example 5

Compounds as described herein may be tested for their potential to inhibit pigmentation of the skin or hair, or to lighten skin or hair, using a minolta meter CM-SA (Konica Minolta Sensing Americas, Inc., Ramsey, NJ; konicaminolta.com/sensingusa/products/Color-Measurement/specialty-meters-med-pers/CM-SA/index.html). Used in combination with a Konica Minolta spectrophotometer, the CM-SA enables highly accurate measurement of skin color simultaneously with a numerical display of the Melanin Index, Hb (Hemoglobin) Index, and Hb SO₂ (Hemoglobin oxygen saturation) Index (%). Measurement will be performed by simply placing the head of spectrophotometer against the skin of the subject and pressing the button. Measurement by just applying light to the face, arm, or other desired part of the body will not put undue stress on the examinees. After a treatment regimen as described in Examples 6-10 below, the melanin index shows lightening of the skin or inhibition of pigmentation. A delta L value (change in the value of L) of 5 points is typically evidence of significant skin lightening.

### Example 6

A simple topical composition may be prepared by combining the following components utilizing conventional mixing techniques:

| Component | Percent by Weight of Composition |
|---|---|
| A compound as described herein | 0.05% |
| ethanol | 85% |

This composition will be applied twice daily to the skin or hair as appropriately needed, in an amount sufficient to deposit about 0.5 μg/cm² skin for six months. The ability of the composition to inhibit pigmentation of the skin or hair and/or lightening of the skin or hair will be determined.

### Example 7

A cream may be prepared by combining the following components using conventional mixing techniques:

| | Percent by Weight |
|---|---|
| Component | of Composition |
| Water phase | |
| U.S. Pharmacopia grade H₂O | 63.03 |
| Disodium EDTA | 0.13 |
| Glycerin | 3.00 |
| Methyl paraben | 0.25 |
| Oil phase | |
| propylene glycol dicaprylate/dicaprate | 3.00 |
| glyceryl stearate | 4.00 |
| cetyl alcohol | 1.00 |
| stearyl alcohol | 1.00 |
| ethoxylated cetyl stearyl alcohol | 1.5 |
| propyl paraben | 0.1 |
| Preservative phase | |
| U.S. Pharmacopia hrade H₂O | 1.49 |
| butylene glycol | 1.50 |
| benzyl alcohol | 0.5 |
| Active solution | |
| A compound as described herein | 0.3 |
| water | 17 |

The first three phases are mixed with the active solution. The composition will be applied to the skin as appropriately needed once every other day for two months to lighten the skin.

Preferred compounds will be tested using this formulation in the pigmented guinea pig to determine their in vivo efficacy in a composition. On each guinea pig from two to six treatment sites (typically 16 cm² each) will be treated topically with compounds formulated in the vehicle (100 μL of 0.1-3% active, 5 times per week for up to 6 weeks) with appropriate placebo and untreated control patches on the same animal. The animals will be visually and instrumentally graded for erythema and skin lightening. Application to a human face (approximately 300 cm²), for example, about 1-2 g (or 1-2 mL) of cream will be used.

### Example 8

A nonionic oil-in-water emulsion may be prepared by combining the following components using conventional mixing techniques:

| Component | Percent by Weight of Composition |
|---|---|
| deionized water | 78.83 |
| propylene glycol | 3.00 |
| octyl methoxycinnamate | 7.50 |
| cetyl alcohol | 2.50 |
| stearyl alcohol | 2.50 |
| laureth 23 | 2.00 |
| C12-C15 alcohols benzoate | 2.00 |
| EDTA | 0.37 |
| methyl paraben | 0.20 |
| propyl paraben | 0.10 |
| A compound as described herein | 1.00 |

The composition will be applied to the skin or hair as appropriately needed once a day for four months. Use of an amount sufficient to deposit about 15 μg of the active per Cm² skin or hair should be appropriate. The ability of the composition to inhibit pigmentation of the skin or hair and/or lighten the skin or hair will be assessed.

### Example 9

A sunscreen composition may be prepared by combining the following components utilizing conventional mixing techniques:

| Component | Percent by Weight of Composition |
|---|---|
| polypropylene glycol 15 | 15.00 |
| stearyl ether | |
| sorbitan oleate | 2.00 |
| octyl methoxy cinnamate | 7.50 |
| propyl paraben | 0.15 |
| butylated hydroxyl toluene | 0.05 |
| cyclomethicone | 20.00 |
| sesame oil | 5.00 |
| mineral oil (Blandol) | 50.30 |
| A compound as described herein | 0.07 |

The above composition will be applied to the skin twice a week for five months. Use of an amount sufficient to deposit 100 μg of the active per cm² skin should be appropriate. The ability of the composition to inhibit pigmentation of the skin and/or lighten the skin will be assessed.

### Example 10

A composition may be prepared by combining the following components utilizing conventional mixing techniques:

| Component | Percent by Weight of Composition |
|---|---|
| deionized water | 89.63 |
| EDTA | 0.37 |
| A compound as described herein | 0.01 |

The above composition will be applied once to the skin or hair as appropriately needed every three days for three months. Use of an amount sufficient to deposit 120 μg of the active per cm² skin or hair should be appropriate to lighten hyperpigmented regions.

### Example 11

Two efficacy and safety studies will be conducted in melasma patients between the ages of 21 to 75 years, having skin phototypes l-IV and moderate to severe melasma of the face. A composition comprising a compound as described herein will be compared with a vehicle composition without a compound as described herein.

The patients will be instructed to apply the composition each night, after washing their face with a mild soapless cleanser, for 8 weeks. The patients will also be instructed to apply a thin layer of the composition to the hyperpigmented area, making sure to cover the entire area including the outside borders extending to the normal pigmented skin. The patients will be provided a mild moisturizer for use as needed and a sunscreen with SPF 30 for daily use. Moreover, the patients will be instructed to avoid sunlight exposure to the face and wear protective clothing.

The patients will be evaluated for melasma severity at baseline and at weeks 1, 2, 4, and 8 of treatment. Primary efficacy will be based on the proportion of patients who had an investigators' assessment of treatment success, defined as a reduction in pigmentation from baseline at the end of the eight-week treatment period.

After 8 weeks of treatment with a composition as described herein, patients will be entered into an open-label extension period in which the composition will be given on an as-needed basis for the treatment of melasma.

### Example 12

Efficacy and safety of any of the compositions described herein for lightening melanocytes in a human skin equivalent MELANODERM™ tissues from MatTek Corp. (Ashland, MA) consist of normal, human-derived epidermal keratinocytes, and melanocytes which have been cultured to form a multilayered, highly differentiated model of the human epidermis. MELANODERM™ B tissue (MEL-300-B), derived from a black donor, will be used for optimal demonstration of efficacy. Tissues will be treated daily with 2 μL of a composition as described herein of the leading skin lightening commercial product, TRI-LUMAS from Galderma, or will be untreated for 7 days. Each day the tissues will be rinsed twice with PBS before the compositions will be reapplied. Compositions will be applied with the end of a glass rod. Tissues will be incubated at 37° C. in a 5% CO₂ atmosphere between composition applications. Photographs will be taken on the 8th day at a magnification of 300× with a light microscope. The melanocytes in MELANODERM™ tissues from the various treatments will be analyzed for the amount of melanin, shape, and number of dendritic processes as well as any other factors that are indicative of normal melanocytes or stressed melanocytes.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of inhibiting pigmentation of hair, skin, or both, the method comprising administering to a subject in need thereof, a safe and effective amount of a compound described herein.

Clause 2. The method of clause 1, wherein the compound is an FP receptor antagonist.

Clause 3. The method of clause 2, wherein the FP receptor antagonist is selected from the group including OBE002, OBE022, and BAY-6672.

Clause 4. The method of clause 2, wherein the FP receptor antagonist is a compound of formula (II), or a pharmaceutically acceptable salt thereof,

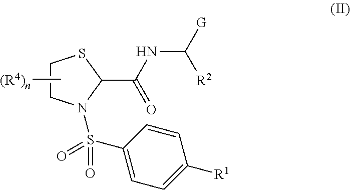

wherein: G is

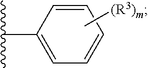

R¹ is tert-butyl or phenyl, wherein the phenyl is optionally substituted with 1-3 R^{1a}; R^{1a}, at each occurrence, is independently C₁₋₄alkyl, C₁₋₄fluoroalkyl, halogen, cyano, —OC₁₋₄alkyl, or —OC₁₋₄fluoroalkyl; R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a}; R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl; R³, at each occurrence, is independently C₁₋₄alkyl, C₁₋₄fluoroalkyl, halogen, cyano, —OC₁₋₄alkyl, or —OC₁₋₄fluoroalkyl; R⁴, at each occurrence, is independently C₁₋₄alkyl or C₁₋₄fluoroalkyl; m is 0, 1, 2, 3, 4, or 5; and n is 0, 1, 2, 3, or 4.

Clause 5. The method of clause 2, wherein the FP receptor antagonist is a compound of formula (III), or a pharmaceutically acceptable sat thereof,

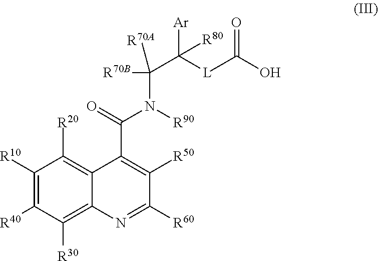

wherein: Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₃₋₄cycloalkyl, —OC₁₋₂alkyl, —SC₁₋₂alkyl, C₁₋₄alkyl substituted by 1-3 fluoro, C₃₋₄cycloakyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy; L is a bond or —X—(CR^{a}R^{b})ₖ—; X is a bond, CH₂, O, S, S(O), S(O)₂, or N(R); R is hydrogen or CH₃; R^{a} and R^{b} are independently hydrogen, fluoro, or CH₃; or R^{a} and R^{b} together with the carbon atom to which they attach form a cyclopropyl; k is 1, 2, 3, or 4; R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro; R²⁰, R³⁰, and R⁴⁰ are independently hydrogen, halogen, CH₃, CH₂F, CHF₂, or CF₃; R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro; R⁶⁰ is —NR^{c}R^{d}; R⁶⁰ is hydrogen or C₁₋₃alkyl; R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃; R^{70A} and R^{70B} are independently hydrogen or CH₃, or R^{70A} and R^{70B} together with the carbon atom to which they attach form a cyclopropyl; R⁸⁰ is hydrogen, fluoro, CH₃, CF₃, CH₂CH₃, or OH; and R⁹⁰ is hydrogen or CH₃.

Clause 6. The method of any one of clauses 1-5, wherein the compound is topically or orally administered to the subject.

Clause 7. The method of any one of clauses 1-6, wherein pigmentation of hair is inhibited.

Clause 8. The method of any one of clauses 1-7, wherein pigmentation of skin is inhibited.

Clause 9. The method of any one of clauses 1-8, wherein the compound is administered for about 6 weeks to about 10 weeks.

Clause 10. A method of lightening skin, the method comprising administering to a subject a safe and effective amount of a compound described herein.

Clause 11. The method of clause 10, wherein the compound is an FP receptor antagonist.

Clause 12. The method of clause 11, wherein the FP receptor antagonist is selected from the group including OBE002, OBE022, and BAY-6672.

Clause 13. The method of clause 11, wherein the FP receptor antagonist is a compound of formula (II), or a pharmaceutically acceptable salt thereof,

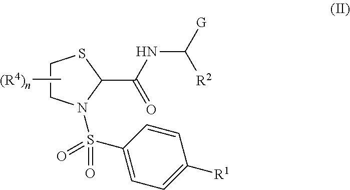

wherein: G is

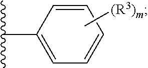

R¹ is tert-butyl or phenyl, wherein the phenyl is optionally substituted with 1-3 R^{1a}; R^{1a}, at each occurrence, is independently C₁₋₄alkyl, C₁₋₄fluoroalkyl, halogen, cyano, —OC₁₋₄alkyl, or —OC₁₋₄fluoroalkyl; R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a}; R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl; R³, at each occurrence, is independently C₁₋₄alkyl, C₄fluoroalkyl, halogen, cyano, —OC₁₋₄alkyl, or —OC₁₋₄fluoroalkyl; R⁴, at each occurrence, is independently C₁₋₄alkyl or C₁₋₄fluoroalkyl; m is 0, 1, 2, 3, 4, or 5; and n is 0, 1, 2, 3, or 4.

Clause 14. The method of clause 11, wherein the FP receptor antagonist is a compound of formula (III), or a pharmaceutically acceptable sat thereof,

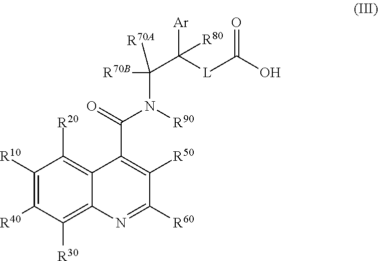

wherein: Ar Is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₃₋₄cycloalkyl, —OC₁₋₂alkyl, —SC₁₋₂alkyl, C₁₋₄alkyl substituted by 1-3 fluoro, C₃₋₄cycloakyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy; L is a bond or —X—(CR^{a}R^{b})ₖ—; X is a bond, CH₂, O, S, S(O), S(O)₂, or N(R); R is hydrogen or CH₃; R^{a} and R^{b} are independently hydrogen, fluoro, or CH₃; or R^{a} and R^{b} together with the carbon atom to which they attach form a cyclopropyl; k is 1, 2, 3, or 4; R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro; R²⁰, R³⁰, and R⁴⁰ are independently hydrogen, halogen, CH₃, CH₂F, CHF₂, or CF₃; R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro; R⁶⁰ is —NR^{c}R^{d}; R^{c} is hydrogen or C₁₋₃alkyl; R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃; R^{70A} and R^{70B} are independently hydrogen or CH₃, or R^{70A} and R^{70B} together with the carbon atom to which they attach form a cyclopropyl; R⁸⁰ is hydrogen, fluoro, CH₃, CF₃, CH₂CH₃, or OH; and R⁹⁰ is hydrogen or CH₃.

Clause 15. The method of any one of clauses 10-14, wherein the compound is administered for about 6 weeks to about 10 weeks.

Clause 16. The method of any one of clauses 1-15, wherein the subject has melasma, chloasma, postinflammatory hyperpigmentation, lentigines, or hyperpigmentation, or wishes to lighten the skin.

What is claimed is:

1. A method of inhibiting pigmentation of hair, skin, or both, the method comprising administering to a subject in need thereof, a safe and effective amount of a compound described herein.

2. The method of claim 1, wherein the compound is an FP receptor antagonist.

3. The method of claim 2, wherein the FP receptor antagonist is selected from the group including OBE002, OBE022, and BAY-6672.

4. The method of claim 2, wherein the FP receptor antagonist is a compound of formula (II), or a pharmaceutically acceptable salt thereof, 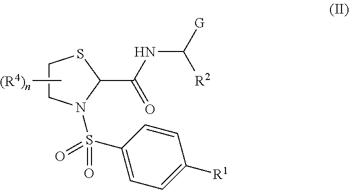
wherein:
g is 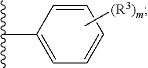
R¹ is tert-butyl or phenyl, wherein the phenyl is optionally substituted with 1-3 R^{1a};
R^{1a}, at each occurrence, is independently C₁₋₄alkyl, C₁₋₄fluoroalkyl, halogen, cyano, —OC₁₋₄alkyl, or —OC₁₋₄fluoroalkyl;
R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a};
R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl;
R³, at each occurrence, is independently C₁₋₄alkyl, C₁₋₄fluoroalkyl, halogen, cyano, —OC₁₋₄alkyl, or —OC₁₋₄fluoroalkyl;
R⁴, at each occurrence, is independently C₁₋₄alkyl or C₁₋₄fluoroalkyl;
m is 0, 1, 2, 3, 4, or 5; and
n is 0, 1, 2, 3, or 4.

5. The method of claim 2, wherein the FP receptor antagonist is a compound of formula (III), or a pharmaceutically acceptable salt thereof, 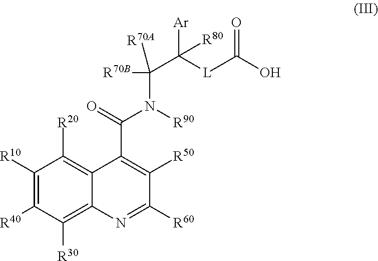
wherein:
Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₃₋₄cycloalkyl, —OC₁₋₂alkyl, —SC₁₋₂alkyl, C₁₋₄alkyl substituted by 1-3 fluoro, C₃₋₄cycloalkyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy;
L is a bond or —X—(CR^{a}R^{b})ₖ—;
X is a bond, CH₂, O, S, S(O), S(O)₂, or N(R);
R is hydrogen or CH₃;
R^{a} and R^{b} are independently hydrogen, fluoro, or CH₃; or R^{a} and R^{b} together with the carbon atom to which they attach form a cyclopropyl;
k is 1, 2, 3, or 4;
R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ are independently hydrogen, halogen, CH₃, CH₂F, CHF₂, or CF₃;
R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ is —NR^{c}R^{d};
R^{c} is hydrogen or C₁₋₃alkyl;
R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} are independently hydrogen or CH₃, or R^{70A} and R^{70B} together with the carbon atom to which they attach form a cyclopropyl;
R⁸⁰ is hydrogen, fluoro, CH₃, CF₃, CH₂CH₃, or OH; and
R⁹⁰ is hydrogen or CH₃.

6. The method of any one of claims 1-5, wherein the compound is topically or orally administered to the subject.

7. The method of any one of claims 1-6, wherein pigmentation of hair is inhibited.

8. The method of any one of claims 1-7, wherein pigmentation of skin is inhibited.

9. The method of any one of claims 1-8, wherein the compound is administered for about 6 weeks to about 10 weeks.

10. A method of lightening skin, the method comprising administering to a subject a safe and effective amount of a compound described herein.

11. The method of claim 10, wherein the compound is an FP receptor antagonist.

12. The method of claim 11, wherein the FP receptor antagonist is selected from the group including OBE002, OBE022, and BAY-6672.

13. The method of claim 11, wherein the FP receptor antagonist is a compound of formula (II), or a pharmaceutically acceptable salt thereof, 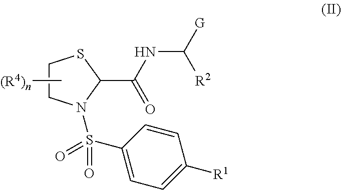
wherein:
G is 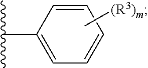
R¹ is tert-butyl or phenyl, wherein the phenyl is optionally substituted with 1-3 R^{1a};
R^{1a}, at each occurrence, is independently C₁₋₄alkyl, C₁₋₄fluoroalkyl, halogen, cyano, —OC₁₋₄alkyl, or —OC₁₋₄fluoroalkyl;
R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a};
R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl;
R³, at each occurrence, is independently C₁₋₄alkyl, C₁₋₄fluoroalkyl, halogen, cyano, —OC₁₋₄alkyl, or —OC₁₋₄fluoroalkyl;
R⁴, at each occurrence, is independently C₁₋₄alkyl or C₁₋₄fluoroalkyl;
m is 0, 1, 2, 3, 4, or 5; and
n is 0, 1, 2, 3, or 4.

14. The method of claim 11, wherein the FP receptor antagonist is a compound of formula (III), or a pharmaceutically acceptable sat thereof, 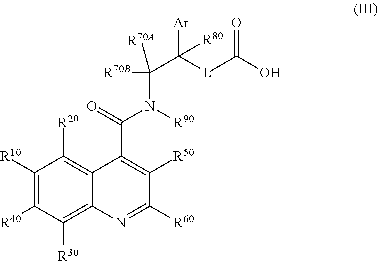
wherein:
Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄alkyl, C₃₋₄cycloalkyl, —OC₁₋₂alkyl, —SC₁₋₂alkyl, C₁₋₄alkyl substituted by 1-3 fluoro, C₃₋₄cycloalkyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy;
L is a bond or —X—(CR^{a}R^{b})ₖ—;
X is a bond, CH₂, O, S, S(O), S(O)₂, or N(R);
R is hydrogen or CH₃;
R^{a} and R^{b} are independently hydrogen, fluoro, or CH₃; or R^{a} and R^{b} together with the carbon atom to which they attach form a cyclopropyl;
k is 1, 2, 3, or 4;
R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ are independently hydrogen, halogen, CH₃, CH₂F, CHF₂, or CF₃;
R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ is —NR^{c}R^{d};
R^{c} is hydrogen or C₁₋₃alkyl;
R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} are independently hydrogen or CH₃, or R^{70A} and R^{70B} together with the carbon atom to which they attach form a cyclopropyl;
R⁸⁰ is hydrogen, fluoro, CH₃, CF₃, CH₂CH₃, or OH; and
R⁹⁰ is hydrogen or CH₃.

15. The method of any one of claims 10-14, wherein the compound is administered for about 6 weeks to about 10 weeks.

16. The method of any one of claims 1-15, wherein the subject has melasma, chloasma, postinflammatory hyperpigmentation, lentigines, or hyperpigmentation, or wishes to lighten the skin.
